(19) United States
(12) Patent Application Publication
(10) Pub. No.: US 2009/0137963 A1
(43) Pub. Date: May 28, 2009

(54) MEDICAL DEVICE HAVING A RETRACTABLE NEEDLE

(75) Inventor: Aaron Rodd, Queensland (AU)

Correspondence Address: CANTOR COLBURN, LLP, 20 Church Street, 22nd Floor, Hartford, CT, 06103, US

(73) Assignee: MEDIGARD LIMITED, Main Beach (AU)

(21) Appl. No.: 12/090,135

(22) PCT Filed: Oct. 16, 2006
(86) PCT No.: PCT/AU2006/001529

§ 371 Date: Sep. 29, 2008

(30) Foreign Application Priority Data
Oct. 14, 2005 (AU) 2005905682

(51) Int. Cl. A61M 5/32

(52) U.S. Cl. 604/195

(57) ABSTRACT

A medical device having a main body, an actuator and a needle, the needle able to be retracted into the main body, the main body having a chamber which is under reduced pressure or can be placed under reduced pressure, the needle attached or attachable to a retraction member movable between a forward position with the needle extending from the main body and a retracted position with the needle within the main body, the retraction member additionally movable between a held position preventing retraction and a release position where rotation of the actuator allows the retraction member to be retracted into the vacuum chamber by the reduced pressure of the chamber.

### FIELD OF THE INVENTION

This invention is directed to a medical device having a retractable needle, and in particular, to a medical device that can reduce needlestick injury by retracting the needle into a vacuum chamber and where the “triggering” of the retraction mechanism comprises a rotating or twisting action. The invention will be described with reference to a catheter/cannula introducer, but it should be appreciated that no particular limitation should be made and the invention is applicable to other types of medical devices that have a needle that may be prone to needlestick injury and which therefore benefit from being retracted in a safe manner. For instance, the invention may also be directed to a medical syringe.

### BACKGROUND ART

In the medical profession, there are many instances where needles are required. The most common instance is with syringes. However, there are other medical devices that contain needles such as catheters/cannulas which usually have a puncture needle (sometimes called an introducer) onto which the catheter/cannula is supported. Other medical devices containing needles may also be available.

Referring to syringes as an example, there are some problems with syringes that do not have a retracting needle. The major problem is the possibility and the risk of needlestick injury, which is of particular concern if the needle is contaminated. Another issue is to reduce the possibility of the needle being reused.

For this reason, there are many examples of retractable needles for medical syringes. The three main types of retractable needles comprise (a) needles that are retracted by vacuum, (b) needles that are retracted by a shoot back spring, and (c) needles that are retracted by a “pull back” spring.

The present invention is directed to retractable needles of the first type; that is, needles that are retracted by being “sucked back” into a vacuum container. The invention is particularly directed to a catheter or cannula introducer which typically comprises a steel needle which can be retracted into a vacuum container which can form part of the device.

The applicant has previous patent applications and granted patents directed to syringes having a plunger which is under vacuum and where the needle is attached to a needle holder which can be sucked back into the plunger after use. The syringe has a tubular syringe body which is open at the rear end. A plunger can be inserted into the open rear end and pushed along the syringe body. The plunger is under vacuum (or can be placed under vacuum upon demand). The front of the plunger (that is the end facing the front of the syringe) is open but is plugged by a seal. The seal is releasably attached to the front of the plunger. The front of the seal contains a special design (claws, fingers, etc). If these fingers, etc. are pushed towards each other, this causes the seal to be released from the front of the plunger and the vacuum in the plunger will cause the seal to be “sucked back” into the plunger.

The front of the syringe contains a needle holder which is releasably attached inside the front of the syringe. The puncture needle is attached to the needle holder. The rear end of the needle holder (facing the plunger) also contains a special profile (claws, fingers, etc,) which holds the needle holder to the front of the syringe.

When the plunger is pushed towards the front of the syringe, the fingers, etc. on the front of the plunger will contact the fingers, etc. on the rear of the needle holder. The fingers, etc. have a special design such that they will engage each other. Thus, the seal on the front of the plunger connects to the needle holder. At the same time, the fingers, etc. are pushed towards each other to release the seal from the plunger and to release the needle holder from the front of the syringe. As the seal and the needle holder are connected together, and as the needle holder supports the needle, this will cause the needle to be sucked back into the rear of the plunger.

A variation of the above is where the needle does not form part of the syringe, but instead forms part of a cannula introducer. In this arrangement, a cannula/catheter (typically a thin somewhat flexible tube) is fitted on to a puncture needle (sometimes called an introducer). The tip of the puncture needle projects from the cannula/catheter. The puncture needle can then be inserted into the person's vein (for example), and once inserted, the cannula can be slowly pushed off the needle and into the vein while the needle is being slowly removed. Typically, a cap or valve is fitted either before or after to the cannula/catheter. Once the needle has been removed, it will be contaminated and will present a definite sharps hazard. Therefore, it is also known to provide a retraction mechanism generally as described above for this type of medical device. Again, the known retraction mechanism can comprise fingers/claws.

The present invention is directed to a retraction mechanism which is somewhat similar to that described above, however the “triggering” of the retraction mechanism is conducted using a twisting or rotating movement as opposed to a forwardly pushing movement. The present invention will find particular use with a catheter/cannula introducer.

It will be clearly understood that, if a prior art publication is referred to herein, this reference does not constitute an admission that the publication forms part of the common general knowledge in the art in Australia or in any other country.

### OBJECT OF THE INVENTION

It is an object of the invention to provide a medical device of the type that has a needle that can benefit from retraction into the device, and where the retraction mechanism will typically use vacuum, and where “triggering” of the retraction mechanism uses a twisting or turning action.

In a broad form, the invention comprises a medical device having a main body, an actuator, and a needle, the needle able to be retracted into the main body, the main body having a chamber which is under reduced pressure or which can be placed under reduced pressure, the needle being attached to, or attachable to a retraction member, the retraction member being movable between a forward position where the needle at least partially extends from the main body, and a retracted position where the needle is substantially or entirely within the main body, the retraction member additionally being movable between a held position where the retraction member is prevented from retracting, and a release position where the retraction member is retracted into the vacuum chamber and by the reduced pressure in the vacuum chamber, the retraction member being movable between the held position and the release position upon rotation of the actuator.

Thus, the medical device may comprise a catheter/cannula introducer which has a main body which can be under reduced pressure and therefore comprise the vacuum chamber, and an actuator that may be fitted to the front of the medical device, a retraction member that may comprise a sliding wall of the vacuum chamber, and a puncture needle that may be attached to the retraction member. The actuator may have some form of operative association with the retraction member such that the retraction member is held or releasably locked to the front of the vacuum chamber but can be released by rotation of the actuator. It is considered that this rotation unlocking retracting mechanism is simpler to use and may be more robust and reliable.

In another form, the invention comprises a medical device, the medical device having an outer body, a chamber which is under vacuum, or which can be placed under vacuum, the front of the chamber containing a seal which may comprise a retraction member, the seal being releasably attached to the front of the chamber and able to be retracted into the chamber under the influence of vacuum but only if the seal has been released from attachment to the front of the chamber, a needle which is attached to or relative to the seal and which can therefore be retracted into the chamber if the seal is retracted into the chamber, release means which may be in the form of an actuator to release the seal from the front of the chamber, the release means comprising a first portion which forms part of the seal, and a second portion which is on the outer body, the outer body and the chamber being rotatable relative to each other, whereby rotation of the outer body relative to the chamber causes the second portion to engage with the first portion and to release the seal from the front of the chamber after which the seal will retract into the chamber under the influence of vacuum in the chamber.

Thus, the contaminated puncture needle can be safely retracted into the chamber simply by relative twisting of the outer body relative to the chamber. There is no need to push hard against the chamber to cause triggering of the retraction mechanism.

In a broad form, the invention comprises a medical device having a first part which may comprise a main body and a second part which may comprise an actuator, at least a portion of one said part being insertable into at least a portion of the other said part, the first part and the second part being rotatable relative to each other, one part comprising a chamber which is under vacuum or which can be placed under vacuum, the chamber containing an open end which is closed by a seal which is releasably attached to the chamber but which can be retracted into the chamber when released, and means to release the seal from the open end of the chamber by rotation of the first part relative to the second part.

Typically, the medical device will have the chamber as the part that slides within the other part. The other part can therefore be seen as the outer body. Typically, the outer body will extend only partially over the chamber and typically will extend only over the forward part of the chamber. However, it is envisaged that circumstances may arise where it is desirable to have the outer body extending substantially along and over the chamber.

The medical device may be made from any suitable material which may include plastic, glass and the like and with possibly larger devices, the device may be made of metal, composite materials and the like. The length of the device may vary to suit and this will depend on the size of the vacuum chamber, the size of the needle and the like. For most circumstances, the medical device will be of the size to enable an “ordinary” length needle to be fully retracted into the vacuum chamber. As an “ordinary” length needle will typically be between 20-100 mm, the length of the medical device will typically be slightly larger. The diameter of the medical device may also vary but it is envisaged that the diameter will typically be between 5-30 mm. Of course, no particular limitation should be placed on the invention merely by providing these sizes.

The chamber (which can also be called the vacuum chamber) will typically be depressurised prior to packing and storing. However, it is also envisaged that the chamber can be depressurised just prior to use (which can also be called “vacuum on demand”).

The chamber will typically be tubular and will typically have an open front end (the front end being closest to the needle) and a closed rear end. It is envisaged that the chamber will be manufactured to be closed at the rear end and to have an open front end. However, the chamber can also be formed as a tube opened at each end and a plug of some form can plug the rear end. The particular manufacture of the chamber should not be limiting to the invention.

The otherwise open end of the chamber can be closed by a seal. The seal may comprise a plug, piston, and the like. The seal may comprise a relatively simple manufacture such as a solid plug that can be retracted when released, or may comprise a more sophisticated manufacture to include a flash chamber. etc. The seal may be made as a unitary body or may be formed from a number of separate parts that can be attached together. The seal may also be provided with some form of indication means to provide a visual indication to the state of the vacuum in the chamber.

The seal is attached to the vacuum chamber and will typically be attached to the otherwise open front of the vacuum chamber. In a particularly preferred embodiment, the seal comprises at least one, and preferably a plurality of fingers that attach over the front edge of the chamber. The fingers can be moved between an attached position where the fingers are attached to the chamber and therefore prevent the seal from being retracted, and a release position where the fingers have been decoupled and the seal can be retracted (under vacuum) into the chamber.

A feature of the present invention is the ability of the seal to be released from the chamber by a twisting or rotating action as opposed to a simple pushing action. In a particularly preferred embodiment, the chamber is within the outer body and relative rotation of the chamber to the outer body causes the seal to be released.

It is preferred that relative rotation causes the at least one finger to move from the attached position to the release position.

It is preferred that the outer body contains at least one projection (for instance a cam or like member) that pushes against the at least one finger and dislodges the at least one finger when the outer body is rotated relative to the vacuum chamber.

### BRIEF DESCRIPTION OF THE DRAWINGS

An embodiment of the invention will be described with reference to the following drawings in which:

FIG. 1 illustrates the view of a medical device containing a puncture needle.

FIG. 2 illustrates an enlarged section view of the medical device of FIG. 1 in the “use” position where the needle has not been retracted and can be used for its purpose.

FIG. 3 illustrates the section view of FIG. 2 and particularly illustrating the components in the “triggered” position where retraction of the needle is just about to happen.

FIG. 4 illustrates a front section view where the seal in the front of the vacuum chamber is in the attached position.

FIG. 5 illustrates the view similar to that of FIG. 4, but now illustrating how relative rotation has caused the seal in the front of the vacuum chamber to become detached.

FIG. 6 illustrates three progressive views showing retraction of the needle into the body of the device.

### BEST MODE

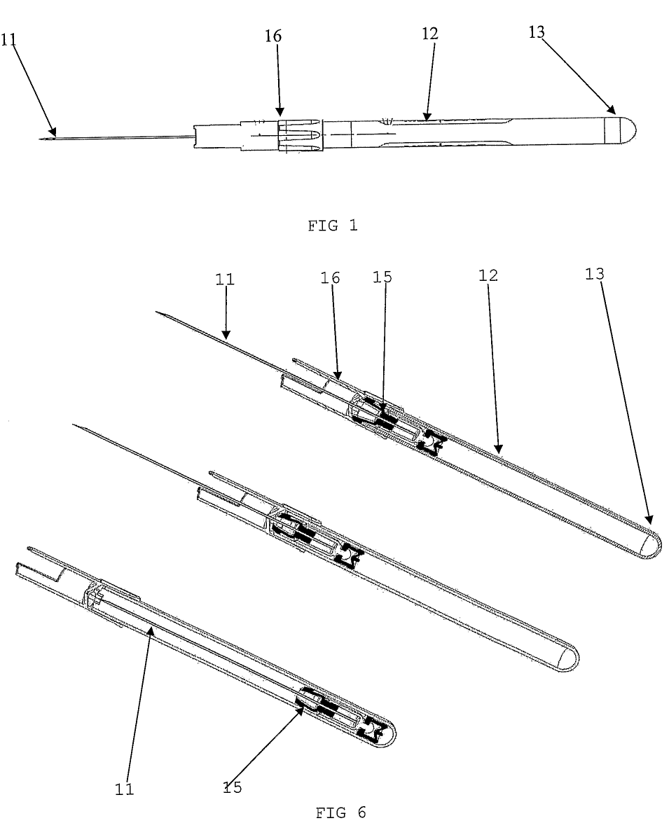

Referring to the drawings and initially to FIGS. 1-5, there is illustrated the main components of the medical device. These components comprise a needle 11, a main body in the form of a vacuum chamber 12 having a closed rear end 13 and an open front end 14 which is initially sealed by a retraction member 15 (also called a seal 15) of rather complicated multipart construction which will be described in greater detail below, and an actuator 16 which is alternatively named the “outer part”. Needle 11 is attached to seal 15. The device contains an outer part 16 (which can also be called outer body or actuator) which extends over the front end of vacuum chamber 12 and can be rotated relative to vacuum chamber 12.

Retraction member/seal 15 forms part of a retraction member to which the needle is attached and which can be sucked back into the vacuum chamber 12.

Briefly, needle 11 can be retracted into vacuum chamber 12 (see for instance FIG. 6) after use by turning outer part 16 relative to vacuum chamber 12. This will be described in greater detail below.

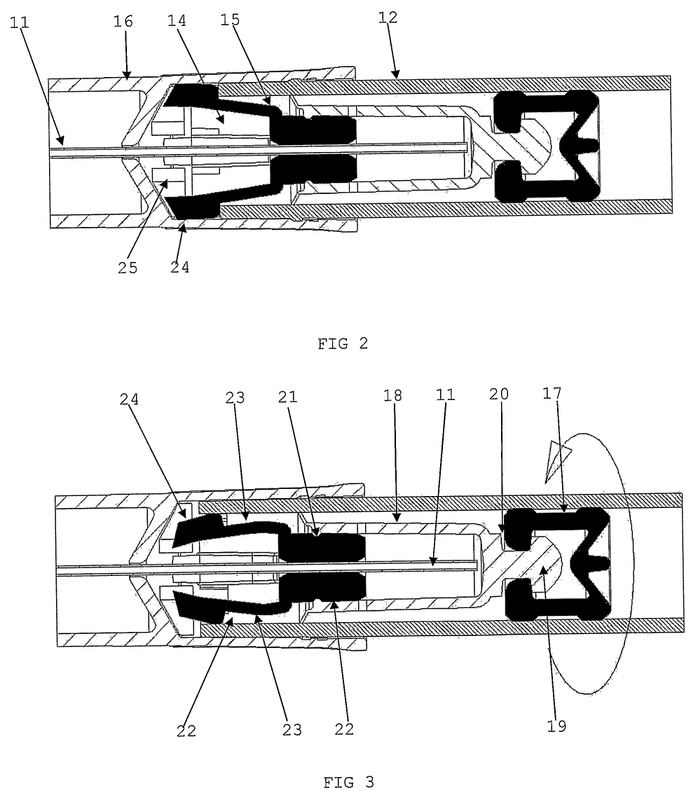

Referring to FIGS. 2-3, outer part 16 extends over and is rotatable relative to the front of vacuum chamber 12. Vacuum chamber 12 has an open front which is plugged by seal 15.

Seal 15 (see FIG. 3) is a multiple part seal and basically comprises a rearmost sealing member 17 which can be made of rubber, silicone, other types of the elastomeric materials and the like. Member 17 is attached to a central main body portion 18 which is made of a more rigid material such as rigid plastics. Main body portion 18 contains a rear head 19 formed with a necked portion 20; this particular arrangement allowing sealing member 17 to be fitted to main body portion 18. Main body portion 18 can be made of clear material to act as a “flash chamber”

The front of the main body portion 18 is fitted with an attachment means 21 which functions to attach seal 15 to the front of vacuum chamber 12. Attachment means 21 comprises a rear portion 22 which plugs into the front of central main body portion 18. The needle 11 is attached to a rear portion 22 via a passageway. Extending forwardly of rear portion 22 is a plurality of finger members 23. Alternatively, a single annular “ring” can be provided. Finger members 23 comprise an end part, or shoulder portion 24 which extends over the front edge of vacuum chamber 12 thereby attaching seal 15 to the front of vacuum chamber 12. However, dislodging finger members 23 from the front edge of vacuum chamber 12 (see for instance the position of finger members 23 in FIG. 3) will release the seal from engagement and will cause the seal to be retracted.

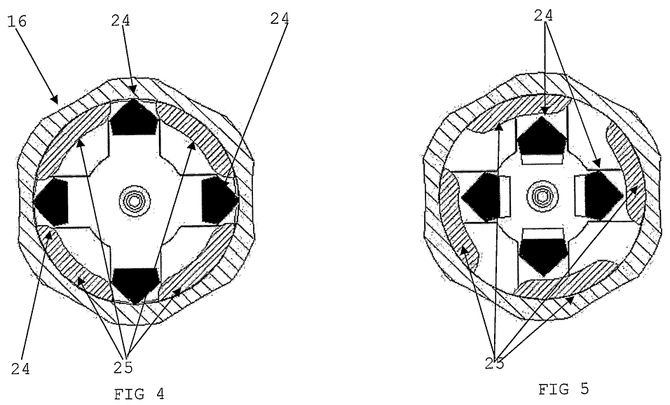

The inside wall of the actuator or outer part 16 contains a plurality (in this case four) internal projections in the form of cams 25. These cams are partially illustrated in FIGS. 2-3, but are better illustrated in the front section views of FIGS. 4-5.

When the finger members (and particularly part 24) of the finger members are attached over the front edge of vacuum chamber 12 (this being the locked position illustrated in FIG. 2 and FIG. 4), the needle cannot be retracted. The positioning of cams 25 (see FIG. 4) is such that there is a spacing between adjacent cams sufficient to allow the finger members to lock to the front of the vacuum chamber 12.

To retract needle 11 after use, the outer body 16 is turned/rotated/twisted relative to vacuum chamber 12 by 45° or thereabouts. When this happens, the cams 25 will push against the finger members and will push the finger members inwardly to release the finger members from engagement with the front of vacuum chamber 12. This is the position illustrated in FIG. 3 and in FIG. 5. As soon as this occurs, the seal is sucked back into the vacuum chamber taking the needle with it, this being progressively illustrated in FIG. 6.

In use, the device will be in the position illustrated in FIG. 1. After use, all that is required is to turn or rotate the body 16 which will cause safe and efficient retraction of needle 11 into the body of the device.

Throughout the specification and the claims (if present), unless the context requires otherwise, the term “comprise”, or variations such as “comprises” or “comprising”, will be understood to apply the inclusion of the stated integer or group of integers but not the exclusion of any other integer or group of integers.

Throughout the specification and claims (if present), unless the context requires otherwise, the term “substantially” or “about” will be understood to not be limited to the value for the range qualified by the terms.

It should be appreciated that various other changes and modifications can be made to any embodiment described without departing from the spirit and scope of the invention.

1. A medical device having a main body [12], an actuator [16], and a needle [11], the needle able to be retracted into the main body, the main body having a chamber which is under reduced pressure or which can be placed under reduced pressure, the needle being attached to, or attachable to a retraction member [15], the retraction member being movable between a forward position where the needle at least partially extends from the main body, and a retracted position where the needle is substantially or entirely within the main body, the retraction member additionally being movable between a held position where the retraction member is prevented from retracting, and a release position where the retraction member is retracted into the chamber and by the reduced pressure in the chamber, the retraction member being movable between the held position and the release position upon relative rotation between the main body and the actuator.

2. The device of claim 1, wherein the main body has a front end, the needle extending from the front end, the actuator being rotatably attached to, or over the front of the main body.

3. The device of claim 2, wherein the front end of the main body is open and the retraction member is releasably attached adjacent the front end of the main body.

4. The device of claim 3, wherein the retraction member includes a sealing member [17] to seal the retraction member to the chamber to prevent loss of vacuum in the chamber.

5. The device of claim 4, wherein the retraction member includes an attachment means [21] to releasably attach the retraction means to the main body.

6. The device of claim 5, wherein the attachment means has at least one shoulder portion [24] that engages with the open front edge of the main body to hold the retention member to the main body.

7. The device of claim 6, wherein the actuator [16] has at least one projection [25] that pushes against the at least one shoulder portion [24] and dislodges the at least one shoulder portion [24] from engagement with the main body when the outer body is rotated relative to the main body, thereby causing retraction of the retraction means and the needle into the main body [12].

8. The device of claim 6, wherein the at least one shoulder portion [24] is at the end of a deformable finger member [23].

9. The device of claim 3, wherein the main body comprises a tube having a front open end and a closed rear end, the chamber comprising the inside of the tube.

10. The device of claim 1, wherein the chamber is placed under vacuum during manufacture of the device.

11. The device of claim 1, wherein the chamber is placed under vacuum prior to use of the device.

12. The device of claim 1, comprising a catheter/cannula introducer.

13. A medical device having a first part which may comprise a main body and a second part which may comprise an actuator, at least a portion of one said part being insertable into at least a portion of the other said part, the first part and the second part being rotatable relative to each other, one part comprising a chamber which is under vacuum or which can be placed under vacuum, the chamber containing an open end which is closed by a seal which is releasably attached to the chamber but which can be retracted into the chamber when released, and means to release the seal from the open end of the chamber by rotation of the first part relative to the second part.
